(19) United States
(12) Patent Application Publication
(10) Pub. No.: US 2023/0320872 A1
(43) Pub. Date: Oct. 12, 2023

(54) METHOD FOR MANUFACTURING PORTIONS OF A PROSTHETIC SOCKET AND KIT

(71) Applicant: ROMEDIS GMBH, Neubeuem (DE)

(72) Inventor: Andreas RADSPIELER, Neubeuern (DE)

(21) Appl. No.: 17/909,703

(22) PCT Filed: Mar. 5, 2021
(86) PCT No.: PCT/EP2021/055610

§ 371 Date: Sep. 6, 2022

(30) Foreign Application Priority Data
Mar. 5, 2020 (DE) 10 2020 106 057.2

(51) Int. Cl. A61F 2/50; A61F 2/76; A61F 2/80; B33Y 50/02; B33Y 80/00; G05B 19/4099

(52) U.S. Cl. CPC A61F 2/5046; A61F 2/76; A61F 2/80; B33Y 50/02; B33Y 80/00; G05B 19/4099; A61F 2002/505; A61F 2002/5053; A61F 2002/763; A61F 2002/7635; G05B 2219/35012

(57) ABSTRACT

The present invention relates to a method for manufacturing or for planning the manufacturing of a prosthetic shaft, of an inner shaft of an outer shaft and/or of an extension of the prosthetic shaft, wherein the prosthetic shaft is provided for receiving a limb stump of a patient. In addition, the present invention relates to a prosthetic shaft and a kit. Furthermore, a computing system, a digital storage medium, a computer program product as well as a computer program are proposed.

### FIELD OF THE INVENTION

The present invention relates to a method for manufacturing a prosthetic shaft or for the planning of manufacturing a prosthetic shaft. The present invention further relates to a prosthetic shaft, inner shaft, outer shaft or to an extension of the prosthetic shaft as well as to a kit with one element from a group consisting of a prosthetic shaft, inner shaft, outer shaft, or at least one extension. In addition, a computing system programmed to carry out the manufacturing or planning method, and a digital storage medium, a computer program product and a computer program to configure a control device and/or a closed loop control device to carry out the manufacturing or planning method are proposed. Furthermore, the present invention relates to a method for creating a data collection relating to data of limb stumps as well as to a set comprising a computing system for executing the data collection creation method.

### BACKGROUND OF THE INVENTION

Leg amputees may regain mobility using leg prostheses. Modern leg prostheses encompass various modules (prosthetic shaft, knee modules, lower leg modules and foot modules), which may be combined to meet the various needs of the prosthesis wearer (hereinafter referred to in short as wearer) in terms of fundamental mobility, sport activities and aesthetic perceptions.

### SUMMARY OF THE INVENTION

The present invention relates to a method which serves preferably for manufacturing or planning the manufacturing or creating of a prosthetic shaft of a prosthesis for, e.g., the lower extremities, e.g., for a leg prosthesis. The prosthetic shaft is the module of the prosthesis, which establishes the connection between the mechanical replacement of the extremity and the residual limb stump (in short also referred to as stump) of the prosthesis wearer, e.g. a thigh stump.

The prosthetic shaft is connected at its distal end (that end facing away from the prosthesis wearer) to a mechanical extremity replacement, in the case of the lower extremity, for example, a modular knee joint-lower leg-foot device for the thigh amputee or a modular lower leg-foot device for the lower-leg amputee. At its proximal end (that end facing the prosthesis wearer), the stump is inserted into the prosthetic shaft. The prosthetic shaft should sit tightly and as form-fit as possible on the stump. The precise fit of the connection with the limb stump determines how securely the prosthesis is held on the stump.

In order to transmit the partly considerable forces between the wearer’s body or stump on the one hand and the prosthesis on the other hand, which occur when standing, walking, standing up, running, etc., a high degree of strength or rigidity of the prosthetic shaft is required. This is ensured by a correspondingly stiff outer section of the prosthetic shaft, the so-called outer shaft. However, since its stiffness causes pressure on the stump, which is regularly perceived as uncomfortable or even painful and can lead to pressure points, the outer shaft is supplemented by a so-called inner shaft (also known as a sleeve) on its inside to increase wearing comfort. The inner shaft usually comprises an elastic material. It may consist of this in the form of an elastic sleeve (liner) or be padded with it.

The object of the present invention may be to optimize the manufacturing of a prosthetic shaft or its planning. Furthermore, a prosthetic shaft and a kit, a computing system, a digital storage medium, a computer program product and a computer program are to be proposed. Furthermore, it is the object of the present invention to propose a method for creating a data collection or for determining measured values. A set is also to contribute to the solution of said object.

The object according to the present invention can be achieved by a method for manufacturing or planning a prosthetic shaft. The object may further be achieved by a prosthetic shaft and the like and by a kit having a prosthetic shaft and the like. Furthermore, a computing system programmed to carry out the manufacturing or planning method, and a digital storage medium, a computer program product and a computer program to configure a control device and/or a closed loop control device to carry out the manufacturing or planning method contribute to achieving the object according to the present invention. A method for creating a data collection relating to data of limb stumps and a set comprising a computing system for executing the data collection creation method serve likewise for achieving the object according to the present invention.

A method for manufacturing or for planning the manufacturing of a prosthetic shaft, an inner and/or an outer shaft and/or an extension of the prosthetic shaft is, therefore, generally or broadly proposed by the present invention. Thereby, the prosthetic shaft is provided in order to receive a limb stump.

The method according to the present invention comprises determining data, in particular geometric data, or providing data, in particular geometric data, already at an early stage, e.g. at a creation timepoint of the prosthetic shaft, of the inner shaft and/or outer shaft and/or of the one or several extension(s). In this, preferably such data is determined, in particular geometric data, which will, e.g. according to experience, assumption, prediction, calculation using models, algorithms, artificial neural networks, artificial intelligence, comparison with a collective and/or machine learning, describe, determine, depict or - because they can hardly ever be complete - co-determine (above terms can be interchanged) the shape of the limb stump at least at one timepoint in the future. Preferably, data or geometric data is determined here which, at least at the first later timepoint, denoted here as wearing timepoint, will describe, determine, depict, co-determine or define the shape of the prosthetic shaft, of the inner shaft or outer shaft or of the extension (or the shape which these should then have). The data or geometric data may be predicted values or measurements, estimated values or expected values, in particular about future shapes. The data or geometric data may have been obtained by the method according to the present invention for creating a data collection or may be based thereon. They may be present in a storage medium, such as a database.

The method optionally further comprises either manufacturing the prosthetic shaft or sections thereof. This is done based on the determined data and/or geometric data. Alternatively or in addition to the aforementioned manufacturing of the prosthetic shaft or sections thereof, the method encompasses creating at least one control signal, in particular creating a control file with control signals, based on which a manufacturing machine may (e.g. directly or indirectly) perform steps for manufacturing the prosthetic shaft, the inner shaft or outer shaft or the extension and/or create at least one of the aforementioned devices or parts thereof.

Thereby, the timepoint of creation (also: creation timepoint) is preferably earlier than the first wearing timepoint.

The method in its general or most general embodiment optionally has no further features.

The prosthetic shaft, inner shaft or outer shaft respectively according to the present invention, or the extension according to the present invention for the prosthetic shaft are manufactured preferably by or using the method according to the present invention.

A kit with at least two elements of a group is also proposed by the present invention. The group consists of a prosthetic shaft, an inner shaft, an outer shaft and at least one extension for the prosthetic shaft. In this, one, two or all elements of the group are created based on the method according to the present invention, that is, e.g. manufactured or produced or created, or possess the properties of such elements.

According to the present invention, a computing system is proposed which is programmed to carry out or prompt each method according to the present invention or the arithmetic steps or computer steps thereof, in particular in interaction with the devices required for this, in particular as disclosed herein. It may comprise a programmable data processing device for this purpose.

A particularly digital, particularly non-volatile storage medium (herein denoted also as carrier) according to the present invention, in particular in the form of a floppy disk, RAM, ROM, CD, hard disk, DVD, USB stick, flash card, SD card, FeRAM or EPROM, in particular with electronically or optically readable control signals, may be configured such that to configure a control device (herein alternatively: closed-loop control device) into a control device with which each method according to the present invention described herein may be effected, or to configure a computing system into a computing system according to the present invention or to prompt a control system or a computing system to carry out any of the methods described herein.

In this, preferably all, several or some of the machine-induced steps of these methods may be prompted.

A computer program product according to the present invention comprises a program code that is volatile or saved on a machine-readable carrier, through which a control device is configured such that each method according to the present invention described herein may be effected, or to configure a computing system into a computing system according to the present invention or to prompt a control device or a computing system to carry out any of the methods described herein.

In this, preferably all, several or some of the machine-induced steps of these methods may in turn be prompted.

The term “machine-readable carrier” as used herein, refers in certain embodiments of the present invention to a carrier, which contains data or information interpretable by software and/or hardware. The carrier may be a data carrier, such as a floppy disk, a CD, DVD, a USB stick, a flashcard, an SD card, an FeRAM, an EPROM or the like.

A computer program product may according to the present invention be understood as, for example, a computer program which is stored on a carrier, an embedded system as a comprehensive system with a computer program (for example, an electronic device with a computer program), a network of computer-implemented computer programs (for example, a client-server system, a cloud computing system, etc.) or a computer on which a computer program is loaded, runs, is saved, executed or developed.

A computer program according to the present invention encompasses a program code by which a control device is configured such that each method according to the present invention described herein may be effected, or to configure a computing system into a computing system according to the present invention or to prompt a control device or a computing system to carry out any of the methods described herein.

In this, preferably all, several or some of the machine-induced steps of these methods may be prompted.

According to the present invention, a computer program may be understood to mean, for example, a physical, marketable software product which comprises a program.

Further proposed by the present invention is a computer-implemented method encompassing the steps herein-mentioned of any method mentioned herein.

Further proposed by the present invention is a data processing device/system comprising means for executing the herein-mentioned steps of any herein-mentioned method.

Further proposed by the present invention is a computer program product encompassing commands which, when the program is executed by a computer, prompt the computer to execute the herein-mentioned steps of any herein-mentioned method.

Further proposed by the present invention is a computer-readable (storage) medium encompassing commands which, when the program is executed by a computer, prompt the computer to execute the herein-mentioned steps of any herein-mentioned method.

The method for creating a data collection according to the present invention encompasses the step of providing a device (herein also: impression device) for use in manufacturing a plaster impression or creating a data model of a limb stump. The device comprises a container or pressure container having a pressure chamber for receiving pressurized fluid, preferably a liquid. The pressure container comprises a wall made of a first material and a fluid-tight membrane made of a second material.

As a further step, the method according to the present invention encompasses providing a pressurization device and/or a pressurization control device for setting, effecting and/or maintaining a pressure prevailing within the pressure chamber.

A further step of the method according to the present invention is a measuring of actual data of the limb stumps, preferably at always the same, predetermined positions or sites of the limb stumps, of a plurality of patients or a collective of patients. The measuring is carried out, respectively, at least at one first measurement timepoint by using the aforementioned device.

The measuring of actual data of the limb stumps respectively at least at one second measurement timepoint, which lies, e.g. days, weeks or months, after the first measurement timepoint, is encompassed as a further step by the method according to the present invention. The measuring is also carried out here by the device and preferably at the same positions or sites of the limb stump as of the measuring carried out at the first measurement timepoint.

For measuring, for example, a measuring device, e.g. of the device, may be used.

The actual data of the limb stumps may be stored in a data collection, e.g. in a suitable storage medium, such as a database, e.g. one referred to herein.

As a further step, the method optionally encompasses an association of patient data (e.g. a health condition or an association of the patient thereto) to the measured actual data which was measured at the first measurement timepoint as well as to at least the measured actual data which was measured at the second measurement timepoint. The measured actual data, which was measured at the at least two measurement timepoints, is thus associated to the same patient data (or the same health condition), respectively.

For example, an association device can be used for associating.

A subsequent optional classification, association and/or evaluation of the actual data measured at the first measurement timepoint and the actual data measured at the second measurement timepoint based on or using the patient data associated to them is also encompassed as a further step by the method according to the present invention.

For example, a classification device can be used for classifying.

The classified actual data is optionally stored, in a further step of the method according to the invention for creating a data collection, based on its classification etc., in a suitable storage medium, such as a database, e.g. the one mentioned previously or another one disclosed herein, e.g. in order to be able to (re)access it within the scope of the method according to the present invention for manufacturing or planning the manufacturing of a prosthetic shaft, inner shaft, outer shaft and/or an extension, which is also provided in some embodiments according to the present invention, for example when determining or providing the geometric data. Alternatively or additionally, the data can be evaluated and/or further processed by suitable devices.

The set according to the present invention comprises a computing system according to the present invention. Alternatively or additionally, it comprises a computing system which is provided and programmed to carry out the method according to the present invention for creating a data collection, in particular by using a provided device or after it has been provided. In addition, the set according to the present invention comprises a device for use in manufacturing a plaster impression or creating a data model of the limb stump, which in turn comprises a container or pressure container having a pressure chamber for receiving pressurized fluid, preferably a liquid. The pressure container here has a wall of a first material and a fluid-tight membrane of a second material.

In all the following statements, the use of the expression “may be” or “may have” and so on, is to be understood synonymously with “preferably is” or “preferably has,” and so on respectively, and is intended to illustrate embodiments according to the present invention.

Whenever numerical words are mentioned herein, the person skilled in the art shall recognize or understand them as indications of a numerical lower limit. Unless it leads the person skilled in the art to an evident contradiction, the person skilled in the art shall comprehend the specification for example of “one” (also “a/an”) as encompassing “at least one”. This understanding is also equally encompassed by the present invention as the interpretation that a numeric word, for example, “one” (also “a/an”) may alternatively mean “exactly one”, wherever this is evidently technically possible for the person skilled in the art. Both understandings are encompassed by the present invention and apply herein to all used numerical words.

Whenever “programmed” or “configured” is mentioned herein, it is then disclosed that these terms are interchangeable.

Whenever an applicability or a method step is mentioned herein, the present invention additionally encompasses also a corresponding programming or configuring of a suitable device or a section thereof, for example, of a computing system and/or its components such as storage medium or storage device for data, e.g. of the collective, calculation device, device for providing geometric data, device for creating a control file, etc. and devices programmed in such a way. The devices, which may be part of, or in signal communication with, the computing system may each be named after the method step they carry out.

When it is disclosed herein that the subject-matter according to the present invention comprises one or several features in a certain embodiment, it is also respectively disclosed herein that the subject-matter according to the present invention does, in other embodiments, likewise according to the present invention, explicitly not comprise this or these features, for example, in the sense of a disclaimer. Therefore, for every embodiment mentioned herein it applies that the converse embodiment, e.g. formulated as negation, is also disclosed.

Advantageous developments of the present invention are each subject-matter of the dependent claims and embodiments.

Whenever an embodiment is mentioned herein, it is then an exemplary embodiment according to the present invention.

Embodiments according to the present invention may comprise one or more of the following features and/or method steps in any combination, unless the person skilled in the art recognizes a specific combination as technically impossible. Also, the subject-matters of the dependent claims specify embodiments according to the present invention.

When the term creating is mentioned herein, it can be understood to mean, when referring to physical objects such as the prosthetic shaft, the inner shaft, etc., as manufacturing, making, fabricating or producing the same. The creation timepoint is then the timepoint of manufacturing, making, fabricating or producing.

In some embodiments, the data is not geometric data, although it is nevertheless perhaps referred to as such.

In several embodiments, the data is topographical data of the limb stump, data reflecting tissue composition, particularly water portion or share, muscle portion, fat tissue portion, or blood flow.

When referring herein to a first, second, or further wear time, it is, in several embodiments, a date that is determinable by the calendar, such as March 1, 30 days from now, 60 days from event X, etc. Alternatively or additionally, when referring herein to a first, second, or further wear timepoint, it may be a timepoint at which a condition has been fulfilled, such as a timepoint at which the prosthesis wearer has walked 5,000 steps, 10,000 steps, or more in the interim, which is countable, for example by a pedometer, or has met some other measure of mobility. Wearing timepoints can also be combinations in which they occur, for example when 10,000 steps have been walked, or when 30 days have passed, or as soon as the first or second of these conditions (and/or further conditions) has been fulfilled, for example when at least 30 days have passed and, in addition, 10,000 steps have been counted in total, etc.

Measurement timepoints may also be determined by walked steps (or by an interval ranging from X steps to Y steps, etc.).

In some embodiments, the present invention or method further comprises reading a pedometer or mobility sensor that records the steps or mobility of the patient, or of the patient while wearing e.g. their prosthesis.

The pedometer or mobility sensor may be integrated in the prosthesis, for example in the prosthesis according to the present invention. The prosthetic shaft, inner shaft or outer shaft, respectively according to the present invention, or the extension for the prosthetic shaft according to the present invention may thus comprise a pedometer or mobility sensor.

The pedometer or mobility sensor may be carried by the patient, e.g. as a wristband, chip, wearable, app on their cell phone, smartphone, or other electronic device.

The pedometer or mobility sensor may be part of a GPS system or a tracking system, etc.

The pedometer or mobility sensor may be connected to a computer, a server, a cloud, a smartphone, etc. by a signal transmitter. It may be connected or in signal communication to a computer of the orthopedic technician caring for the patient or to a computer in a workshop for prosthesis construction or shaft construction, to a shaft milling machine or the like.

A signal receiver may be prepared to evaluate, read out or similar the signals from the signal transmitter. Received signals can be evaluated such that, on the basis of their information content or their structure, it is determined when, for example, a first, second or further wearing timepoint has been reached or is likely to be reached. Thus, it can be determined, for example, by the computing system that the next wearing timepoint, which should be reached when 10,000 steps have been walked or taken with the current prosthetic shaft, inner shaft, outer shaft or extension, is approaching. Thus, a prosthetic shaft, inner shaft, outer shaft or extension as described herein may be created, or a control file for the creation thereof, may be created in good time so that the next required (replacement) prosthetic shaft, inner shaft, outer shaft or extension may be created for the patient without re-measuring their limb stump, which said patient can fetch or collect or pick up, for example, after a corresponding notification or have it delivered by mail, all of which is also encompassed by the present invention.

In several embodiments, the data or geometric data determined in the method according to the present invention, above all for the purpose of manufacturing or for the planning of manufacturing, is predicted data. It is then not actual data and/or measured data representing or co-determining the shape of the limb stump at the timepoint the data is determined or at the timepoint the prosthetic shaft or control signals are created. A prosthetic shaft manufactured on the basis of such data would not fit the patient optimally at the timepoint of creation, but only later, at the timepoint referred to here as the first wearing timepoint. However, such measured data may have been used for prediction. The predicted data may be or may comprise variable dimensions of the limb stump and/or data which is subject to changes over time, for example postoperatively or as a result of a previous surgical operation. The circumference or the water portion (edema) of the limb stump is mentioned here as an example. The predicted data may already reflect changes in the limb stump since the creation of the prosthetic shaft, inner shaft, etc. In other words, by comparing the data or dimensions of the limb stump which it actually comprises at the timepoint of creation (“actual data”) with the data which it comprises after the elapse of time between the timepoint of creation and the first or further timepoint of wearing or will comprise according to or based on experience, a change in shape which has taken place in the meantime can be read off.

In some embodiments of the method according to the present invention, determining (which may in some embodiments be understood as in the sense of using) the data and/or geometric data, which is predicted data for the specific or concrete patient, takes into account patient data reflecting the, in particular current, health condition of the patient. Predicted data and patient data may or will be associated with or linked or assigned to each other.

“Patient data” as used herein may reflect the health condition, e.g. at the moment of determining or creating, or may encompass together, in any combination, the patient’s age as well as further medical characteristics or particularities mentioned herein or may consist thereof. They may, in addition or alternatively, provide information about medical characteristics that were present prior to or at the timepoint of determination or creation, such as pre-existing diseases. Such patient data may be measured and/or anamnestically or clinically collected, etc.

Examples of this patient data may include:
the patient’s age or age group
information on the time elapsed since the amputation,
whether the fitting of the patient with a prosthesis is a fitting with a first or with a subsequent prosthesis,
whether the patient is obese (measurable, for example, by body mass index (BMI) or by bioimpedance, e.g., by an electronic scale, body composition monitor, and the like),
the genesis or cause of the amputation (for example, as a result of peripheral arterial occlusive disease (PAOD), of a trauma or inflammation and/or the presence of a tumor, inflammation and/or the like),
whether the patient has a peripheral arterial occlusive disease,
cardiac performance data (for example, whether the patient has heart failure),
whether the patient has a protein deficiency,
whether the patient suffers from diabetes (melliuts),
whether there is an insufficiency of the patient’s lymphatic system (for example, preor postoperative lymphedema),
the patient’s venous situation (for example, the presence of deep vein thrombosis (DVT) or other thromboses and/or existing varicose veins (varices) and/or whether the patient has already undergone varicose vein surgery, for example),
operations already performed (in particular in the area of the extremity affected by the amputation, e.g. in the groin due to a cardiac catheter operation),
heart failure,
metabolic diseases,
movement behavior of the patient (athlete, walker, office worker without sport balance, etc.),
how many steps the patient takes in a unit of time (hours, days, weeks, etc.),
step size or length,
walking or running speed, e.g., average, maximum, mobility profile throughout the day, etc.,
mobility measured by ways other than pedometers,
composition of body tissue, e.g. of the limb stump, by regarding the liquid, fat and/or muscle content (e.g. by a bio-impedance measurement, e.g. measured by a body composition monitor) or information about a content thereof (in absolute values), information about the blood circulation, information about the distance over which the limb stump protrudes over bony structures (e.g. femur), e.g. in their longitudinal direction,
body mass index,
relationship between body surface of a defined area of the limb stump and the patient’s body weight;
medication of the patient, in particular long-term medication.

Patient data may herein include e.g. a health condition or an association of the patient to one or to several health conditions, which may include the age of the patient but also their medication, physical activity, number of steps taken, etc. as mentioned herein.

This patient data preferably does not include the fact that the patient is missing at least one limb or parts of an extremity.

Geometric data, in several embodiments, thus optionally includes or is associated with patient data in addition to a geometric statement, preferably at least in a stored state of the data, such as information about a pre-existing health condition or underlying disease (see above examples of patient data), and/or is categorized, classified, or cataloged based on the associated patient data.

Thus, for example for a patient P, to be cared for, who is 40 years old and diabetic, data of a collective of patients who were also 40 years old and diabetic when data concerning their limb stump were collected with them may be considered.

However, in several embodiments, geometric and/or predicted data is not reference data which is e.g. available in a data collection or library comprising sample designs for prostheses or sections thereof, such as subdivided with respect to skeletal structure of the cross-section (i.e. relating to how far a bony structure is located from a skin surface of the limb stump), with respect to limb length, and tissue mass, especially if such reference data is not further categorized or classified with respect to further patient data (see, e.g., above). In some embodiments, such reference data is particularly not taken into account if it should be used to create a prosthetic shaft, inner shaft, outer shaft, extension, etc. which fits or fits best at the creation timepoint, and which should fit or should fit as best as possible when immediately used (and not only at the later wearing timepoint).

Thereby, the data, e.g. the patient data, may be taken, used and/or processed individually or in any combination, for example in the presence of diabetes and obesity and/or in connection with a certain age or age group, in order to predict changes in the shape of the limb stump over time (e.g. between the creation timepoint and the first wearing timepoint) and thus to determine the geometric data. In particular, grading of findings or staging of the aforementioned findings and other pre-existing diseases may be taken into account and included in the prognosis, e.g. as patient data.

An option or a measure with which such patient data may enter into the determination of the geometric data may be an input or information in percent, centimeters, or in another manner or - purely optionally - in other units. This measure may lead to a change in the prognosis data determined, for example, from a collective of patients, as exemplarily shown in FIG. 6, or may be incorporated into them, for example, by subtracting empirical values known from observation of diabetics or patients with other medical conditions relevant to future changes in the shape of the limb stump (such as those mentioned, for example, above). Likewise, such a measure may, purely optionally, need to be added to the collective data for some specific medical conditions. The present invention also encompasses a percentage factor (or its use) by which the empirical data of the collective of patients whose patients do not have the specific medical condition can be multiplied.

According to the present invention, the collective data may have been collected from patients who are not affected by the concrete medical condition. This data may be adapted as described above to the specific patient with the specific medical condition, e.g. by using the above-mentioned measure.

However, in some embodiments, the collective data has already been collected from patients who have the one and/or the other of the above-mentioned medical conditions (patient data) or another medical condition, or from those who have any combinations of two or more such medical conditions. In this case, no adjustment is necessary. The data, collected in the collective as prognosis data for the care of the coming patient P, generally do not need to be adapted to the medical condition, since this was also present in the collective. It may suffice here to classify or categorize the collective data according to its patient data. However, an adjustment may still be made optionally, either because it may be necessary or advantageous. Thus, it may be advantageous to optionally consider the degree to which the medical condition is or was present (staging, stage, degree, classification, NYHA, age, etc.) for fine adjustment.

The data collected on the collective and used as prognosis data for the patient P who is awaiting care can of course be classified taking into account the time interval, e.g. between the amputation or the creation timepoint on the one hand and the first wearing timepoint on the other. Alternatively or additionally, for a specific patient P who is 40 years old and diabetic, the patient collective data concerning 40 year old diabetics may in some embodiments, be used to predict how their limb stump will change between the creation timepoint and the first wearing timepoint (in e.g. one week, two weeks or 3 months, etc.). With regard to the patients of the collective and the data collected on them over a sufficiently long period of time, it can thus be said for the specific patient P at the creation timepoint, for example that their limb stump will probably lose e.g. 4 cm in circumference, experience a volume reduction of e.g. 5%, or similar.

The actual data of the collective, taking into account the associated patient data of the measured patients, may be stored, e.g. in one of the devices according to the present invention, or in a location where the devices according to the present invention, e.g. the computing system, can access them, such as in a database, a storage device, the computing system itself, etc. The devices according to the present invention may be configured to (re)access such stored data. Alternatively, such data may be entered by the user of the devices according to the present invention by an input device provided and/or configured for this purpose. The optionally required comparisons between patient data of the specific patient and a collective, if provided, can optionally run automatically, for example once, several times and/or regularly.

In some embodiments of the method according to the present invention, the patient data is taken into account when determining the data or geometric data, e.g., by mathematical calculation operations.

If reference is made herein to mathematical calculation operations, such as addition, subtraction, division or multiplication, all other known mathematical calculation operations are also encompassed by the present invention. The ways mentioned above and below for determining the prognosis data or for determining or adapting the geometric data, in particular due to medical conditions, are purely exemplary.

In several embodiments, the actual data of the collective that is or was stored, is or was collected using a device which serves in manufacturing a plaster impression or creating a data model of the limb stump. Such a device, also referred to herein as an impression device, may comprise a pressure container having a pressure chamber for receiving pressurized fluid, preferably a liquid. The pressure container may comprise a wall made of a first material. The wall defines an interior of the pressure container relative to an exterior. The pressure container may comprise an insertion opening for inserting the limb stump into the interior of the pressure container. Such a device may comprise a fluid-tight membrane made of a second material arranged to form or limit the fluid chamber or the pressure chamber. The pressure container may comprise a device for adjusting the pressure prevailing in the pressure chamber.

In some embodiments, the actual data of the patients is, or was, advantageously collected by such an impression device, since in this way they can be collected mechanically, so to speak, or at least using a machine and thus reproducibly. In this way, it is advantageously possible to measure a, possibly also smaller, number of patients or their limb stumps, and to arrive at results (actual data or measured values) that are clearly comparable with each other. Deviations, or at least relevant deviations, between the procedure for collecting actual data on the limb stump of a first patient A of the collective on the one hand and the procedure for collecting actual data on the limb stump of a second patient B of the collective on the other hand are not to be expected by or according to the present invention, since the manual procedure, which can depend on the orthopedic technician in charge and moreover on his experience, is replaced or at least significantly supported and standardized by the use of the device.

Likewise, for the same reason, deviations, or at least relevant deviations, between the procedure for collecting actual data on the limb stump on the first patient A of the collective at a first timepoint on the one hand and on the same patient A at a second timepoint on the other hand are not to be expected by or according to the present invention.

In some embodiments, the actual data determined in, or for, the method according to the present invention are, or comprise, values measured on the limb stump of different patients of the collective, which were collected for the considered patients of the collective at different measurement timepoints, e.g., several times at a constant interval (e.g. every four weeks) or at different intervals, e.g. with an increasing interval between successive times.

In several embodiments, the aforementioned actual data of the collective is, or was, collected with or under an increasing pressure on the limb stump during the course of the number of collection timepoints. Thus, when the aforementioned impression device is used, the pressure that prevails in the pressure chamber during the said collection, is set by the machine side or is or was at least once applied to the limb stump between a first measurement or collection timepoint and a subsequent measurement timepoint, may be increased. The other settings on the impression device may remain unchanged. The observation made by the inventor of the present invention has shown that in order to produce a prosthetic shaft which fits optimally at the timepoint of the collection or measurement, with an increasing interval, e.g. to the initial fitting of this patient after amputation, increased pressure must be applied to the limb stump at least in the following weeks or months in order to determine the dimensions of a prosthesis which fits optimally at this timepoint.

The aforementioned impression device for collecting the actual data of the patients of the collective may be designed as described in WO 2016/135320 A1, WO 2018/065362 A1, or WO 2018/234486 A1. Reference is hereby made in full to their disclosures.

In several embodiments, the data, in particular the geometric data, describe the shape of the limb stump KS of the specific patient P treated using the method according to the present invention at a timepoint in the future (“future data”) and, hence, differ from the data which describes the limb stump KS of this patient P at the timepoint of creation (“actual data”). A prosthetic shaft, inner shaft, outer shaft or extension created with or according to the “future data” would not yet fit the patient at the timepoint of creation and/or would not be manufactured according to the data or dimensions available at the timepoint of creation (and should not yet fit the patient at the timepoint of creation, but will fit the patient at a later wearing timepoint), since the limb stump will change its shape between the timepoint of creation and the wearing timepoint.

In some embodiments, the data or geometric data determined in the method according to the present invention is or comprises the result of an estimation. Alternatively or in addition, they are read from a reference source. Such a reference source may be, for example, a table of values and/or a database.

Estimating and/or reading and/or calculating may be done by a device, calculation device, or the like programmed for this purpose.

In several embodiments, the actual data that is or was collected from a collective of patients and is used according to the invention as geometric data for the specific patient P would or will be collected by means of or using a device or impression device that serves for the manufacturing a plaster impression or creating a data model of the limb stump. Such a device comprises a pressure container having a pressure chamber for receiving pressurized fluid, preferably a liquid. The pressure container has a wall made of a first material and a fluid-tight membrane made of a second material. The device serves to at least partially receive the limb stump of the patient who is to be fitted with a prosthesis in order to determine the shape of the limb stump, as far as helpful for fitting a prosthesis.

In several embodiments, the geometric data is or comprises the result of a calculation based on an algorithm. This algorithm may in turn be created by expert systems and/or may be based on a plurality of comparative data. In this, measured actual data may be used and/or already processed data, for example data which has been already classified or assigned to patient data, may be further processed.

Both reference source and algorithm may be the result of or constitute the use of artificial intelligence. For example, machine learning tools, e.g. based on artificial neural networks, may have been or are used to determine or process the geometric data based on the actual and/or patient data provided, in any of the ways known to the skilled person.

The geometric data may have been obtained by evaluating measurement results on limb stumps of a plurality of patients, wherein the number of patients or the collective may preferably be 10, 50, 100, or more, preferably per class or category. For obtaining the geometric data, e.g. the actual data of the respective limb stump of the individual patients from the plurality of patients may have been measured, such as the cross-sections of the limb stump at a distance of, for example, 6 cm, 9 cm, and 12 cm from a reference point or cross-section. These measurement results may be compared (as actual data) with the measurement results of the same patient obtained at later times or later wearing timepoints, for example after 3 months and after 6 months. This means that the course of the change in the shape of the limb stump of these patients over the various measurement timepoints is known, e.g. at t=0, i.e. for example immediately after amputation, i.e. for example in the context of initial fitting of the respective patient with a prosthesis, at the first wearing timepoint or at another measurement timepoint of the respective patient after perhaps one month, at a second wearing timepoint or measurement timepoint after perhaps a further month, at a third wearing timepoint after perhaps a further four weeks, i.e. after a total of three months, etc.). In this, the actual data and measurements obtained in this way may be collected depending on further patient data, for example in a reference source, and/or may be evaluated. If actual data of the patient P or their limb stump KS to be fitted by the orthopedic technician is now known, it may be taken from the reference source or another storage location or determined from it how, based on the experience gained from the measured values/actual data on the limb stumps of the plurality of patients of the collective, the geometric data of the limb stump KS of this patient P will presumably be at one wearing timepoint or several later wearing timepoints (e.g. after 1, 2, 3, 4, 5 and/or after 6 months), since on the basis of the collective it can be estimated how the limb stump KS will change, since it is known how the limb stumps of the patients of the collective have changed over time (e.g. after 1, 2, 3, 4, 5 and/or after 6 months). The empirical values collected from the patient collective can, therefore, be used to predict how the shape of the limb stump of the specific patient P fitted with a prosthesis at the timepoint of creation, for example, will probably be at later wearing timepoint, in particular at the reference points considered, e.g. measurement points. Interpolations and extrapolations can be helpful here.

Optionally, the aforementioned reference source and/or the aforementioned algorithm are thereby based on actual data or measured data and provide or output data that often deviates in terms of size from these, for example as geometric data.

In some embodiments of the method according to the present invention, at least 30 days, preferably at least 90 days, in particular at least 180 days, lie between the timepoint of creation of the prosthetic shaft or the timepoint of creation of the control file for manufacturing it on the one hand and the first wearing timepoint of the prosthetic shaft on the other hand.

In certain embodiments, it is thereby irrelevant whether, for example, a prosthetic shaft produced according to the present invention is actually worn at the first wearing timepoint. Rather, it is important that it was manufactured so that it can be worn at the first wearing time, in particular of course with the highest possible wearing comfort on that day. This increased wearing comfort may be the aim of the method according to the present invention.

In several embodiments of the method according to the present invention, the geometric data additionally comprises data which describes, determines or - because it can hardly ever be complete - will co-determine (herein also briefly just: co-determine) the shape of the limb stump (e.g. according to experience, assumption, prediction, calculation using a model, algorithms, artificial neural networks, artificial intelligence, comparison with a collective and/or machine learning) at least at a second timepoint, herein referred to as second wearing time. This data may take into account a change of the limb stump over time, for example the change of its dimensions (e.g. by swelling or shrinking). The second wearing timepoint lies after the first wearing timepoint, wherein at least 3 days, preferably at least 10, 20, 30, 60, 90 days, in particular at least 180 days, 12 months or 24 months, lie between the two wearing timepoints.

In some embodiments of the method according to the present invention, a prosthetic shaft, an inner shaft or outer shaft and/or at least one extension is also created in the step of creating. Alternatively or additionally, a control file containing control data may also be created. In these embodiments, the prosthetic shaft and/or the corresponding control file is based on the geometric data determined, measured and/or calculated with the method according to the present invention and which will co-determine the shape of the limb stump at least at a second wearing timepoint as described above.

It is therefore possible by the present invention to determine both geometric data for the first wearing timepoint as well as, preferably at the same time, that is e.g. on the same day, in the same session, during the same visit of the patient at the orthopedic technician (or vice versa), geometric data for a second wearing timepoint and/or further wearing timepoints and/or to create a prosthetic shaft, inner shaft or outer shaft or extension for the first wearing timepoint and for the further wearing timepoint(s).

In certain embodiments, an extension is an addition or supplement to the prosthetic shaft, an accessory, a built-in element, an inner shaft, or the like.

In some embodiments, the extension is provided to be arranged inside the outer shaft, the inner shaft, or the prosthetic shaft in general, e.g., at or from the first or further wearing time. It may preferably be given to the patient P to take home on its creation day, wherein the day of creating may correspond to the day of determining.

In several embodiments, e.g., of the method or kit according to the present invention, the inner shaft serves to receive the limb stump or at least sections thereof. In this, it is intended to be received in turn, at least in sections thereof, in an interior of the outer shaft.

In some embodiments, e.g., of the method according to the invention, the extension of the prosthetic shaft is an inlay, a pad, a pressure insert, a compression insert, a stocking with different wall thicknesses or a double-walled stocking with at least one insert that is inserted between its layers.

The extension is not adjustable in some embodiments. It may optionally be depressible; it may optionally be elastic. It is not adjustable in some embodiments; optionally, it may, or will, for example, be non-inflatable, not or will not be connected to a pump, have no lines or be connected thereto, and/or not be variable by the patient.

In some embodiments, the prosthesis or the prosthetic shaft does not comprise a device for actuating the extension, for example, a mechanical device provided for this purpose such as an actuator, a pump, a fluid reservoir and/or lines provided for actuating the extension.

In several embodiments, the computing system is configured to be in signal communication with, or is in signal communication with, a manufacturing machine.

When a signal communication or communication connection between two components is mentioned herein, this may be understood to mean a connection that exits during use. It may also be understood that a preparation for such a (wired, wireless or otherwise implemented) signal communication exists, for example by coupling both components, for example by pairing, etc.

Pairing is a process that takes place in connection with computer networks in order to establish an initial link between computer units for the purpose of establishing the communication. The best-known example of this is the establishing of a Bluetooth connection, by which various devices (e.g. smartphone, headphones) are connected to each other. Pairing is sometimes also referred to as bonding.

In some embodiments, the computing system is part of a manufacturing machine or vice versa.

In several embodiments, e.g., of the method according the present invention, the manufacturing machine for creating the prosthetic shaft or sections thereof is a printer, a 3D printer, a molding device, a milling machine, a rapid prototyping device, a CNC milling machine, a CAD milling machine, a thermoforming device, or an injection device. Here, the manufacturing machine is optionally configured to create the prosthetic shaft, inner shaft, outer shaft, or extension (or respective sections thereof) based on control signals. These control signals may in turn have been created by the method according to the present invention.

In some embodiments, the inner shaft and outer shaft are joined together, for example, by joining methods such as gluing, riveting, and the like.

The inner shaft may be dimensionally stable. It may be non-elastic. Its shape may, without wanting or needing to destroy it, be dimensionally stable or solid, at least under normal conditions of use for it or for the prosthetic shaft according to the present invention. The inner shaft may consist of or comprise carbon fibers.

The inner shaft may consist of the same material as the outer shaft or comprise the same material as the outer shaft.

The inner shaft may have an individualized circumferential shape. The individualization may consist of adapting its shape to a circumferential contour of the outer shaft.

The outer shaft may have a first strength or elasticity (expressed, for example, as total strength or total elasticity, or as average total strength or total elasticity, for example in the direction of greatest extension of the circumferential section). The inner shaft may have a second strength or elasticity that is higher than or equal to the first, that is, higher than or equal to that of the outer shaft, using the same procedure for measuring the strength or elasticity.

In several embodiments, the outer shaft surrounds the inner shaft only in sections, while in others it surrounds the inner shaft completely.

In certain embodiments, the inner shaft is preferably at least one of the following: one-piece, circumferentially closed, circumferentially seamless without doubling in the sense of wrinkling, without step, without gap and/or slit, no elastic liner. In some embodiments, this also applies to the outer shaft.

In some embodiments, the outer shaft, the inner shaft and/or the extension(s) comprise at least one connection device by which at least two of the aforementioned components may be connected to each other.

The connection device may encompass or consist of at least one Velcro connection, an adhesive connection and/or a screw connection.

In several embodiments, the extension is not or does not comprise a pumping system.

In some embodiments, the outer shaft and/or the inner shaft are not flexible.

In several embodiments, the outer shaft and/or the inner shaft do not have a lacing system or a tensioning system that would serve to change the volume surrounded by the respective shaft or to change the diameter of the respective shaft or shaft section.

In some embodiments of the kit according to the present invention, a first element of the group, consisting of a prosthetic shaft, an inner shaft or outer shaft or at least one extension of the prosthetic shaft, was created based on data or geometric data, which preferably co-determine the shape of the limb stump at the first wearing timepoint. In addition, a second element of the group was created based on data or geometric data, which preferably co-determine the shape of the limb stump at the second wearing time.

Optionally, at least any element of this group was created at the timepoint of creation, based on the available actual data of the specific patient P, which reflect or co-determine the shape of the limb stump of this patient at the timepoint of creation. Such actual data may be or have been measured, for instance by the impression device disclosed herein for use in creating a data model or manufacturing a plaster impression, for example, directly on the limb stump. Normally, it does not correspond to the determined data or geometric data.

In some embodiments, the step of determining the geometric data does not comprise (or does not correspond to) any of the steps that lead to obtaining measurement data of the specific patient P that is obtainable when measuring the limb stump or that would have been measured at said patient’s limp stump, at least not in order to already create on this basis the prosthetic shaft, outer shaft, inner shaft or extension or control signals to be used at the first wearing timepoint and created for this purpose according to the present invention. Steps leading to the obtaining of measurement data lead in particular to data obtained by measurement by measuring tape, by scanning, by laser measurement or laser scanning, by ultrasound measurement or ultrasound scanning, by determination of the proportions of solid tissue (bone) in relation to the proportions of soft tissue (fat, edema, connective tissues, muscles).

In several embodiments, the step of determining also encompasses considering actual data or measured data, referred to herein as measurement data. Obtaining the measurement data (as actual data) from the specific patient P, however, is optionally not part of the method.

With the aid of such actual data, at least one element of the group consisting of prosthetic shaft, inner shaft, outer shaft or at least one extension may be generated or has been generated. This means that at the timepoint of creation, the patient may be given both an element of this group which already fits them initially (based on their actual data), and at least one further element of this group which will only fit them at the first wearing time, which may be weeks in the future (based on the determined geometric data).

In some embodiments, the method according to the present invention comprises particularly no ultrasonic measurement.

Determining, in several embodiments, is understood to mean reading out, estimating, predicting, and/or specifying geometric data for a future wearing timepoint, preferably however it is not to be understood as a measuring at the specific patient P done at said wearing timepoint.

In some embodiments, the method according to the present invention does not encompass a scanning step, a sonographic step, and/or a creation of a bone model.

In several embodiments, the method according to the present invention does not encompass the consideration of a so-called reduction measure (RM) or, in addition, it does. One speaks of a reduction measure when the orthopedic technician determines or specifies, for example, from the circumference of the thigh and from a constriction measurement, a dimension by which the dimensions of the shaft to be manufactured by the orthopedic technician must deviate from the measured actual data measured on site at patient P, so that the shaft manufactured by the orthopedic technician provides sufficient support for patient P when immediately afterwards using said shaft and when it is loaded with said patient’s body weight. The reduction measure is, therefore, taken into account by the orthopedic technician for dimensioning and designing the shaft of the prosthesis, with the aim of ensuring that the prosthetic shaft is to be as optimally as possible adapted to the measured limb stump and its properties at the timepoint of its first use (creation timepoint). Taking into account a reduction measure thus serves to compensate for inadequacies that occur in connection with the measurement of the limb stump before the patient uses the prosthesis for the first time.

In several embodiments, the method also encompasses creating control signals or a control file with control signals, each for creating a prosthetic shaft although this is not done based on the determined geometric data, but based on measured actual data. The actual data reflects the shape of the limb stump at the timepoint of creation, while the data or geometric data describes or approximates the shape of the limb stump at one or more wearing timepoint(s) lying more or less far after the creation timepoint.

In some embodiments, the geometric data is approximated data that deviates from data that would have been measurable or was measured on patient P at the creation timepoint or at a particular wearing timepoint.

In several embodiments, the geometric data is not data of the specific patient P and is not data collected from the patient, but rather data collected from a patient collective or using a patient collective, or data calculated based thereon.

When it is said herein that the geometric data will co-determine the shape of the limb stump at a future wearing time, this means in several embodiments that there is a reasonable assumption that this will be the case. This assumption may be based on empirical values. It may be based on the fact that, based on the specific shape of the stump at the creation timepoint and values determined, for example, from patient collectives, it can be assumed that the limb stump with sufficient experience will show the geometric data at the targeted wearing time, such as a predicted circumference at a predetermined height, for instance measured at a predetermined distance from, for example an immovable bony structure. It is not certain whether this will happen.

Should measuring take place at further measurement timepoints, the pressure is preferably increased several times or constantly between two successive measurement timepoints.

Preferably, all of the actual data mentioned herein, in particular data of both the specific patient P and of the patients in the collective, is or will be measured, optionally at any timepoint using the same impression device or sensor arrangement (or with identical impression devices or sensor arrangements), or during the respective limb stump which is to be measured was placed in the impression device filled with water. Preferably, there is, or was, pressure prevailing or being applied on the limb stump of the patients of the collective, which pressure was always calculated and/or set in the same way, wherein the pressure or how it was determined and/or set - across the collective - may differ over the timepoint of creation, the moment of initial fitting, the different wearing timepoints or measurement timepoints, but preferably not between the individual patients of the collective respectively at the same wearing timepoint (i.e., e.g., always after 1 month, or e.g., always after 1 year). e.g. always 1 month, or always 2 months, or...) after creation timepoint, measurement timepoint, moment of initial fitting, etc. This may result in a highly valuable reproducibility, which may also correspond to the pressure applied to the prosthesis by the patient’s weight at the respective wearing timepoint.

If, for example, a pressure in the device is set for the specific patient P at the creation timepoint in a manner x (e.g. calculated by applying the same formula, always at a constant value y for the pressure, etc.), it is then provided in some embodiments that, for the purpose of manufacturing or planning the manufacturing of their prosthetic shaft, etc., data or geometric data was determined or provided, wherein this geometric data has been collected from a collective of patients whose limb stumps, at their creation timepoint or timepoint of initial fitting, have been/were also subjected to a pressure in the device which pressure was set in the same manner x (e.g. calculated by applying the same formula, always at a constant value y for the pressure, etc.). The patients selected in this way, who may also be matched with the specific patient P in terms of their patient data (same age, same disease, etc.), may be those whose subsequent measurements are considered as the basis for the geometric data for the first wearing time, the second wearing time, etc., when manufacturing or planning the manufacturing of the prosthetic shaft, etc., of the specific patient P.

Measurement timepoints, as used herein, preferably do not fall on a day specified by date or calendar, but depend on a predetermined event, such as the day of the amputation of the limb that left behind the limb stump, or the day the newly amputated patient first left the bed, or the day the patient’s limb stump was first measured for manufacturing a prosthesis (creation timepoint), etc. Likewise, a measurement timepoint may be understood as a measurement period of manageable length, such as a period of time covering, for example between 3 to 10 days after amputation, etc. In this way, the measurement timepoints may all depend on, or be calculated from, the same event but in the lives of different patients in the collective. If they are for example each calculated from the day of amputation, the first measurement timepoint may be e.g. one month after amputation, the second measurement timepoint may be e.g. two months after amputation, and so on. This means that, for example the first measurement timepoint does not fall on the same day in the calendar for all patients, but has the same time interval for patient A from their amputation day as for patient B or at least falls in a time period of predetermined length for A and B.

If (first, second, etc.) wearing timepoints are now determined for the specific patient P in order to determine geometric data of the collective of patients associated with these wearing timepoints, the actual data measured on the collective at the first, second, etc. measurement timepoint is used for this purpose. If one is interested e.g. in data which the limb stump KS will encompass or show at a first wearing timepoint which is, e.g. one month after the amputation or after the creation timepoint, then one orients oneself to geometric data which was available or measured as actual data in the collective one month after the comparable event (amputation, creation timepoint, etc.).

A “measurement timepoint” as used herein may be identical to a “wearing timepoint”. If a wearing timepoint (for a specific patient) is, for example, 30 days after the amputation data can be used that was collected at a measurement timepoint that was also 30 days after the amputation (in the collective).

The terms “measurement timepoint” and “wearing timepoint” may therefore be interchanged in some embodiments.

In some embodiments of the method for creating a data collection, for the purpose of measuring the actual data, the pressure prevailing in the pressure chamber is set and/or maintained lower on the device using the pressurization control device, when measuring at the first measurement timepoint than when measuring at the second measurement timepoint.

In some embodiments, the first measurement timepoint is the timepoint of initial fitting or of creation.

In several embodiments, the pressure that should be present in the pressure chamber during a measurement timepoint is determined using a reference table or using a simple or simplified formula.

Such a formula may be

$$\text{P} \text{=} \left(\text{m*9,} \text{81*4*} \text{Π}\right) / {\text{U}}^{2}$$

with:
P as the target pressure or set pressure to be set by the pressurization control device,
m as the mass of the patient (possibly weighted by weight),
Π as Ludolph’s Constant, and
U as the circumference of the limb stump, e.g. in the region of the insertion opening or the insertion plane of the prosthetic shaft and/or the most distal (with the patient standing in the initial position according to the neutral zero method) horizontal cross-section of the limb stump, also known as the insertion plane.

The above-mentioned value 9.81 may be understood as a location factor and/or may be exchangeable for it and may have the dimension m/s².

This formula leads to sufficiently good results in a first approximation. However, it may be modified. Other quantities mentioned herein may also be used.

As an alternative formula, P = F/A is optionally used by the present invention, with
P as target pressure or set pressure,
F as the force acting on the membrane, e.g. as the patient’s weight (possibly weighted or not), and
A as the surface of the limb stump subjected to the pressure P during measurement within the impression device.

If A decreases with increasing time since e.g. the amputation with e.g. a stable patient weight G over e.g. successive measurement timepoints, then P must be increased to cause a constant force acting on the limb stump during measurement.

P = G/Q is optionally used by the present invention as a further formula, with
P as target pressure or set pressure,
G as the ‘patient’s weight (possibly weighed down with weights or not), and
Q as the cross-sectional area of a cross-section of the limb stump; the cross-sectional area may be that of the most distal (with the patient standing in the initial position according to the neutral zero method horizontal) cross-section of the limb stump, also known as the insertion plane.

If Q decreases with increasing time since e.g. the amputation with e.g. a stable patient weight G, then P must be increased to cause a constant force acting on the limb stump during measurement.

In some embodiments, therefore, a constant product of P*Q or P*A or one that does not move beyond a predetermined range of values (e.g. expressed as a percentage, such as a maximum of +/- 2%, 3%, 5%, 10%, or 15%) is sought over some or all of the successive measurement timepoints, or is effected by pressure adjustments using the pressurization device or the pressurization control device.

In a simple case, there may be aimed for a constant product of P*U² that does not move beyond a predetermined range of values (e.g. expressed as a percentage, such as a maximum of +/- 2%, 3%, 5%, 10% or 15%) over some or all of the successive measurement timepoints, or to bring this about by pressure adjustments using the pressurization device or the pressurization control device.

In some embodiments, for the purpose of measuring the actual data, e.g. relating to the specific patient at the creation timepoint and/or relating to the collective of patients (preferably all patients and at all measurement timepoints) at the measurement timepoints, the product which is or which results from the pressure prevailing in the pressure chamber during measurement on the one hand and the circumference, the square of a circumference or the cross-sectional area of the limb stump, preferably in the insertion plane, when measured at the first measurement timepoint and when measured at the second measurement timepoint and/or at further measurement times, on the other hand, is constant or lies within a predetermined range of values. This product may optionally vary from patient to patient. It should preferably be constant with respect to the individual patient.

Circumference, square of a circumference, or cross-sectional area of the limb stump may refer, for example, to the heights shown in FIG. 6 at 12 cm or 15 cm (not drawn, see column A in FIG. 6).

In several embodiments, none of the methods of the present invention encompasses user interaction with a 3D shaft or limb model in order to modify it. In some embodiments, none of the devices according to the present invention is configured to enable such user interaction.

In some embodiments, the computing system and/or impression device have transmitting or receiving devices in order to be able to be in signal communication with each other using them. The transmitting or receiving devices may be prepared, configured and/or programmed for this purpose. Actual data measured or collected by the impression device may thus be transmitted, preferably automatically, to the computing system, e.g. by cable or wirelessly, e.g. through a computer-implemented computer programs (e.g. client/server system, cloud computing system, etc.).

In certain embodiments, the impression device and/or set comprises a stocking, adherent stocking, or other sensor arrangement to be pulled over the limb stump, wherein the stocking, adherent stocking, or sensor arrangement comprises sensors for creating a data model or for transmitting positional data.

In some embodiments, measuring actual data encompasses, when creating the data collection, using a stocking, adherent stocking with sensors, or other sensor arrangement to be pulled over the limb stump, wherein the stocking, adherent stocking, or sensor arrangement comprises sensors for creating the data model or for transmitting position data.

The sensors may be arranged at a smaller distance from each other in a section lying more distally on the limb stump than in a section lying proximally thereto. The distance may refer to the circumferential direction and/or the longitudinal direction of the limb stump.

In several embodiments according to the present invention, the device comprises at least one, preferably two or more, cameras, imaging systems, surface scanners, magnetic or laser scanners, 3D scanners, infrared scanners or other types of scanners, ultrasound devices or other devices which are suitable and/or configured to detect or measure the adherent stocking or the sensor assembly, into which the limb stump is inserted, or sensors thereof, and/or to determine the volume and/or geometry (e.g., length, width, surface, outer contour, radii, curvatures, dents, edges, angles, and the like) thereof, or of the limb stump. Based on these measurements, detections, scans and similar, it may be possible to manufacture a shaft for the limb stump without having to make a plaster impression for this purpose. Advantageously, this procedure according to the present invention does not require the manufacturing of a plaster impression. In certain embodiments according to the present invention, the measurement is a scanning and/or a surface scanning of the limb stump.

In some embodiments according to the present invention, the surface of the limb stump is scanned with at least one sensor, advantageously with at least one line structure of sensors, e.g. with sensors arranged in a line or row, which are arranged, e.g., along one or several straight lines.

In several embodiments according to the present invention, the sensors are arranged on a surface, e.g., of the adherent stocking or the sensor arrangement. The surface is optionally designed to conform to the surface of the limb stump for measurement.

In some embodiments according to the present invention, the surface may consist of, or comprise, band-shaped sections. The band-shaped sections may intersect or overlap, for instance at a midpoint. The midpoint may be equidistant or substantially equidistant from the free ends of the band-shaped sections meeting at the midpoint.

The space (alternatively: gap) between two band-shaped sections meeting each other at or near a midpoint and/or being adjacent to each other may be free, i.e. in particular free of material. The space (or gap) may increase in width towards the free end or towards the, in use, proximal or upper end of the band-shaped sections. In this way, a folding of the sensor arrangement or of the adherent stocking may be advantageously counteracted in the case of a slim limb stump, or a folding may even be completely avoided.

In several embodiments according to the present invention, the surface consists of or comprises a preferably even number, for instance six, eight or ten, band-shaped sections. Any two of them may optionally merge to one piece in the region of the midpoint.

In some embodiments according to the present invention, the width of the band-shaped sections is 1 cm to 5 cm, preferably exactly or about 2 cm.

In several embodiments of the present invention, the band-shaped sections are not elastic.

In some embodiments according to the preset invention, some or all of the band-shaped sections, in particular band-shaped sections being adjacent to each other, are connected to each other by further, preferably elastic, band-shaped sections or other, again preferably elastic structures.

In several embodiments according to the present invention, the band-shaped sections are releasably connected to the further band-shaped sections or other structures.

In some embodiments according to the invention, the connection between the band-shaped sections and the further band-shaped sections or other structures comprises or is a hook-and-loop bond.

In several embodiments according to the present invention, the band-shaped sections or the other structures are non-releasably connected to the further band-shaped sections.

In several embodiments according to the present invention, the band-shaped sections or other structures are glued to the further band-shaped sections.

In several embodiments according to the present invention, the band-shaped sections are riveted to the further band-shaped sections or other structures.

In several embodiments according to the present invention, the band-shaped sections are together with the further band-shaped sections or other structures at an angle to each other, which is between 70° and 110°, preferably at a right angle, approximately measured during intended use.

In some embodiments according to the present invention, the band-shaped sections preferably extend completely or substantially along the longitudinal axis of the limb stump or in a projection thereon and/or along the outer contour of the limb stump in a direction from distal to proximal when the sensor arrangement is used as intended.

In several embodiments according to the present invention, all or some of the sensors are iron cores, preferably with magnetic coils, or comprise iron cores, preferably with magnetic coils.

In some embodiments according to the present invention, the sensors and/or the transmitting device and/or the receiving device are not based on an optical system or a laser-based system.

In some embodiments according to the present invention, the device or set comprises a transmitting device and/or a receiving device for transmitting and/or receiving signals from the sensors of the adherent stocking or the sensor arrangement.

In some embodiments according to the present invention, the sensor arrangement, the sensors, the transmitting device and/or the receiving device are designed and/or used as disclosed in WO 2018 234486 A1, the disclosure of which is hereby fully incorporated by reference.

Methods disclosed herein may constitute a common method. Their steps may be carried out in any combination in a common method.

In several embodiments, the impression device or its pressure container comprises a wall having or made of metal. In some embodiments, the membrane comprises silicone. In certain embodiments, the membrane is directly or indirectly screwed to the pressure vessel or clamped therewith using one or several screws.

One or more of the advantages mentioned above or below may be achieved by some or all of the embodiments according to the present invention.

The limb stump is subject to daily volume fluctuations, as well as to partly significant volume changes in the months following the amputation, initially due to postoperative edema and scarring and later due to muscular atrophy.

It is desirable that the prosthetic shaft fits as if freshly fitted to the limb stump at both the moment of creating or fitting it (the creating time) and later times (the relevant wearing timepoints) and still provides the required stability even after muscular atrophy, especially when standing and walking. It is obvious that, for example, a prosthetic outer shaft, which must provide considerable stability to fulfill its task, cannot adapt to such a change in the cross-section of the limb stump due to its strength or firmness.

The present invention advantageously serves to provide rapid adjustment or adaptability of the prosthetic shaft to a change in shape of the limb stump. The adjustment may be conveniently performed by the wearer himself by changing the accuracy of fit of his prosthesis in a few simple steps. For example, the wearer only has to insert the extension or the inner shaft into the prosthetic shaft and fix it there, if provided (screwing, clicking in, gluing, etc.), which, based on the data determined for later use, namely for use at the first (or a further) wearing timepoint, may already have been manufactured ahead of time at the timepoint of creation.

Since the wearer may easily adapt the prosthetic shaft, e.g. manufactured on the day of creation, to a change in the shape of the limb stump by increasing the accuracy of fit of the prosthesis given to him, e.g. at the timepoint of creation, e.g. by inserting the extension at the first wearing timepoint, it is possible and desirable for the prosthetic shaft to fit as well as possible from a technical point of view on the day it is created. The prosthetic shaft may thus be manufactured to fit the limb stump geometry present at this moment optimally from the first minute of wear (e.g. already at the creation timepoint and thus well before the first wearing timepoint). A deterioration of the “fit” in the course of time (e.g. over the coming weeks) due to changes in the shape of the limb stump is not further problematic in the light of the present invention, since the patient may find the optimum fit of the prosthetic shaft from a technical point of view without having to visit the orthopedic technician again. The patient himself may restore the optimum fit of the prosthetic shaft from the point of technical point of view, e.g. by inserting the second inner shaft given to them at an early stage (e.g. at the timepoint of manufacture) or by inserting the extension given to him at an early stage, which is already adapted to the expected change in shape of the limb stump up to the first timepoint of wearing. However, since this is so easy for him to do, and since his prosthesis shaft already fits perfectly from the first minute of wear, i.e. already at the timepoint of creation, he also wears it as intended from the first minute of wear, and in particular he puts a regular load on it. The latter contributes to the fact that the limb stump is loaded from the first minute of wear as desired by the doctor and that the prosthetic shaft is in optimal mechanical interaction with the limb stump from the first minute of wear, whereby the limb stump preferably does not develop, for example, pressure points, scarring, wound healing disorders, cornification, etc. due to a prosthetic shaft that does not fit optimally from the first minute of wear. Such developments of the limb stump, which regularly become chronic, can be advantageously avoided by the present invention, or the probability or severity of their occurrence can be reduced. Since the prosthetic shaft created at the creation timepoint of fits the patient as well as possible from the first minute of wear, it is advantageously possible to mobilize the patient sufficiently early and comprehensively, which is advantageous insofar as otherwise no sufficiently rapid recovery takes place, for example with regard to the healing of amputation wounds on the limb stump.

The use of the same impression device or a device always identical in construction for this purpose when measuring on the collective and on the specific patient, as proposed in some embodiments, may significantly increase the reproducibility of the data obtained and the value of the data obtained on the collective for the specific patient, also because the influence and individual experience of orthopedic technicians, which may each lead to different measurement results, may be replaced by an objective procedure. This is further supported if the pressure in the impression device during measurement, as disclosed herein, is or was always set in an identical manner at specific measurement or wearing timepoints.

### BRIEF DESCRIPTION OF THE DRAWINGS

The present invention is in the following exemplarily explained based on the accompanying drawings, in which identical reference numerals denote the same or similar components. The following applies in the partially highly simplified figures:

FIG. 1 shows a prosthetic shaft as part of a thigh prosthesis being only partially shown with several extensions according to the present invention in a longitudinal section;

FIG. 2 shows a cross-section of the thigh prosthesis of FIG. 1;

FIG. 3 shows an inner shaft according to the present invention as part of a thigh prosthesis in a longitudinal section, in which the thigh prosthesis is only partially shown;

FIG. 4 shows a cross-section of the thigh prosthesis of FIG. 3;

FIG. 5 shows an exemplary embodiment of the computing system according to the present invention;

FIG. 6 shows schematically a reference source for use in the method according to the present invention;

FIG. 7 shows a first embodiment from the side of a section of a longitudinally cut device or impression device used according to the present invention having a pressurization control device;

FIG. 8 shows a method according to the present invention for manufacturing or for planning the manufacturing of a prosthetic shaft, an inner shaft, an outer shaft and/or an extension of the prosthetic shaft, schematically simplified in an exemplary embodiment; and

FIG. 9 shows a method according to the present invention for creating a data collection, schematically simplified in an exemplary embodiment.

### DETAILED DESCRIPTION

FIG. 1 shows an outer shaft 4 as part of a prosthetic shaft 2 of a thigh prosthesis being only partially shown. The relatively stiff, shell-shaped outer shaft 4 receives in its interior a preferably comparatively flexible inner shaft 6 which is inserted removably and which is individually adapted to the limb stump of the patient P.

The optionally closed distal end 8 of the outer shaft 4 is followed by a column-like component 10 leading to the mechanical knee joint (not shown in FIG. 1).

Unlike prostheses of this type, as known from the prior art, here - e.g. between the longitudinally extending walls of the outer shaft 4 and the inner shaft 6 - e.g. two extensions 12 and 14 according to the present invention are arranged, which may each press the wall 18 of the inner shaft 6 inwards by their inner wall in the relevant areas in order to achieve a local reduction in the internal volume of the shaft.

An optional, further such extension 20 according to the present invention is located on the outside of the inner shaft 6 at its distal end.

More precisely, the extensions 12 and 14 are optionally arranged here in the dorso-lateral area following an edge 22 of the thighbone (femur) 24 (indicated by dashed lines) or in the medial-distal area. In this, the extension 12, extending from proximal to distal, optionally has an elongated shape, whereas the extension 14 optionally has a rather round shape.

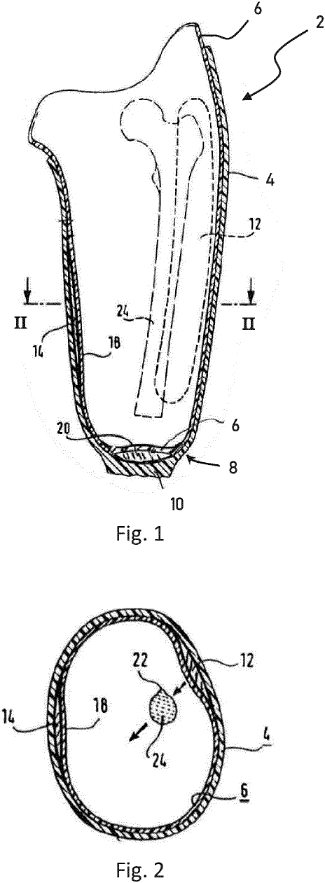

As indicated by the arrows in the cross-section through the prosthetic shaft 2 of FIG. 1 shown in FIG. 2, the femur 24 intentionally undergoes a more or less strong adduction as a result of the extension 12. This allows the abduction, that usually occurs in transfemoral amputees some time after the amputation, to be corrected. In addition, the extensions 12 and 14 allow the shaft volume to be reduced and provide the residual limb with increased surface adhesion in the shaft, here: in the inner shaft 6. This surface adhesion in turn makes it possible, with the aid of the optional extension 20, to restore a desired residual limb end contact after swelling has subsided and, if necessary, after atrophy processes.

The extensions 12, 14 and 20 have been given together with the outer shaft 4 to the patient P, on whose limb stump the outer shaft 4 was adapted on the day of its manufacturing (that is, for example, at the creation timepoint).

It was not necessary to use of the extensions 12, 14, 20 on day of handing over the prosthesis with the outer shaft 4, nor would they have provided the patient P with increased wearing comfort. According to the present invention, however, it had already been determined on or before the day of handing over (e.g., at the timepoint of determination) how some of the data or geometric data of the limb stump would in all likelihood change in the foreseeable future (i.e., at the first wearing timepoint). Up to a day, referred to herein as the first wearing timepoint, the limb stump had changed due to muscular remodeling and possibly a reduction in swelling such that the outer shaft 4 produced at that time could no longer fit optimally. Patient P can independently correct the deviation between the changed shape of their limb stump and the unchangeable shape of the outer shaft 4 of their prosthesis by inserting the extensions 12, 14, 20, which were determined for them while taking into account the changes of the shape (according to geometric data) of their limb stump expected in their case. In the present example, the patient has only to insert the extensions 12, 14 and 20 as already envisaged by the orthopedic technician at the determining timepoint and, if necessary, secure them against slipping within the prosthetic shaft 2. In this way, the patient can restore the desired accuracy of fit for their prosthesis without having to visit the orthopedic technician again and without expert knowledge.

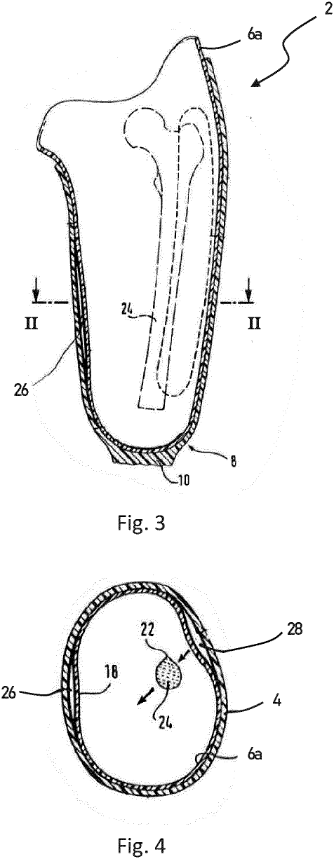

FIG. 3 shows a second embodiment of the prosthetic shaft 2 in longitudinal section. FIG. 4 in turn shows a cross-section thereof.

Unlike what is shown in FIG. 1, the prosthetic shaft 2 comprises no extensions 12, 14 or 20. The muscular remodeling and also the decrease in possible post-operative edema are compensated for by the special design of the inner shaft 6a, which differs fundamentally from the inner shaft 6 of FIG. 1 and FIG. 2.

At the sites at which in the embodiment of FIGS. 1 and 2 extensions 12 and 14 were exemplarily provided to compensate for muscular remodeling with the aim of exerting pressure on the femur 24 in the direction of the arrow, in the embodiment shown in FIG. 3 and FIG. 4, the rigid inner shaft 6a shaped in a special way takes over or adopts this function. Its rigidity results in the formation of empty spaces 26 and 28. They have the shape of the extensions 14 and 12 of FIGS. 1 and 2, respectively. The rigidity of the inner shaft 6a in FIG. 3 allows it to remain form-stable while still exerting the desired pressure on the thigh stump.

In this, the inner shaft 6a is, so to speak, a shaft from the retort: Its dimensions are not based on the actual dimensions or on dimensions that the orthopedic technician measured on the limb stump of the patient P in order to provide said patient P with a prosthesis. Rather, its dimensions are based on data predicted into the future or on geometric data with the expectation at the timepoint of the fitting that the limb stump would assume or adopt said predicted data or geometric data later and, hence, were determined before or at the creation timepoint.

In or according to the embodiment shown in FIGS. 3 and 4, it is thus assumed in the present example that the inner shaft 6a is already the second inner shaft, i.e. an inner shaft that was intended to be worn only at, or starting from, the first wearing timepoint. It is assumed that the orthopedic technician fitted the patient with an inner shaft, not shown in the figures - which may likewise be part of the kit according to the present invention -, which was placed inside the outer shaft 4 with continuous contact to the inside thereof. It is further assumed that this original inner shaft no longer fitted optimally at a first wearing time, e.g. weeks or months after being provided by the orthopedic technician, which is why it was replaced, at the first or at a later wearing time, by the inner shaft 6a, shown in FIGS. 3 and 4, while retaining the original outer shaft 4.

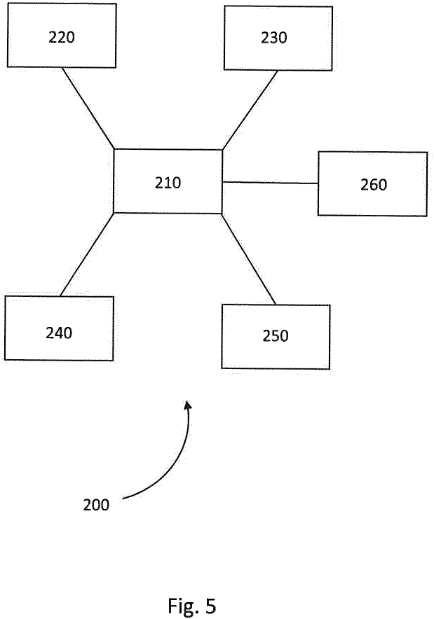

FIG. 5 shows a computing system 200 according to the present invention.

The computing system 200 optionally comprises a calculation device 210, a reference source 220 (for instance a database), an input device 230, an output device 240, a manufacturing machine 250 and/or a step counter or mobility sensor 260, respectively. The aforementioned units 220, 230, 240, 250 and 260 are each optional and may be connected to or integrated with the calculation device 210. They may be in one-way or two-way signal communication with the calculation device 210. They may be interconnected in any manner. Each of these connections may be wired or wireless.

The calculation device 210 may serve to determine the geometric data. For this purpose, it may make use of an optional reference source 220 in which reference data may be stored. For example, by specifying the actual dimensions measured at the creation timepoint, optionally supplemented by other data or patient data such as the age, weight, mobility classification (1 to 4), physical activity, etc. of the patient P, which may optionally be entered via the input device 230, by simply associating this data with empirical values of already provided geometric data, which the limb stump KS of the patient is likely to assume at certain times in the future (referred to herein as wearing timepoints) may be output. The output may be done via the output device 240, e.g. in the form of a notification on a display or as a printout for the orthopedic technician. In addition to or instead of an output, control signals (individually or as part of a control file) may be transmitted to the manufacturing machine 250. The desired component, for example the inner shaft or the extension, or sections or parts thereof, can be produced or manufactured on it, optionally automatically. The indication and/or control signals may encompass information as to where, for example, produced extensions 12, 14, 20 are to be placed in the prosthetic shaft 2.

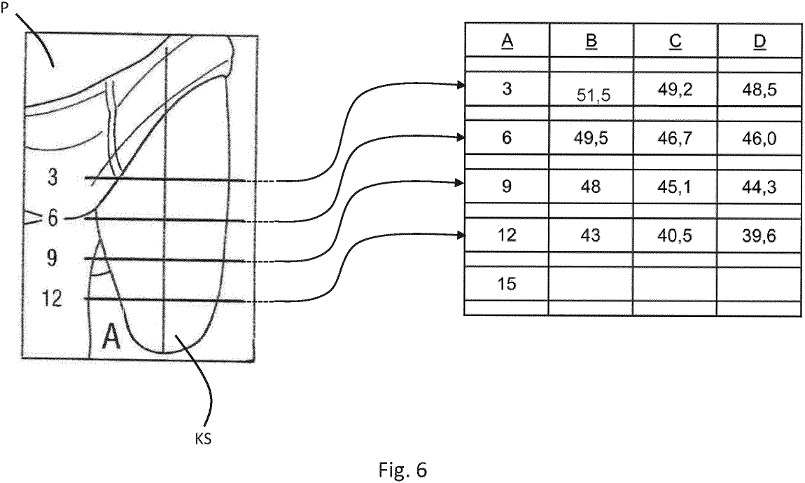

FIG. 6 shows an example of how measured actual data (optionally in connection with patient data) of a collective is used to determine geometric data of the specific patient P.

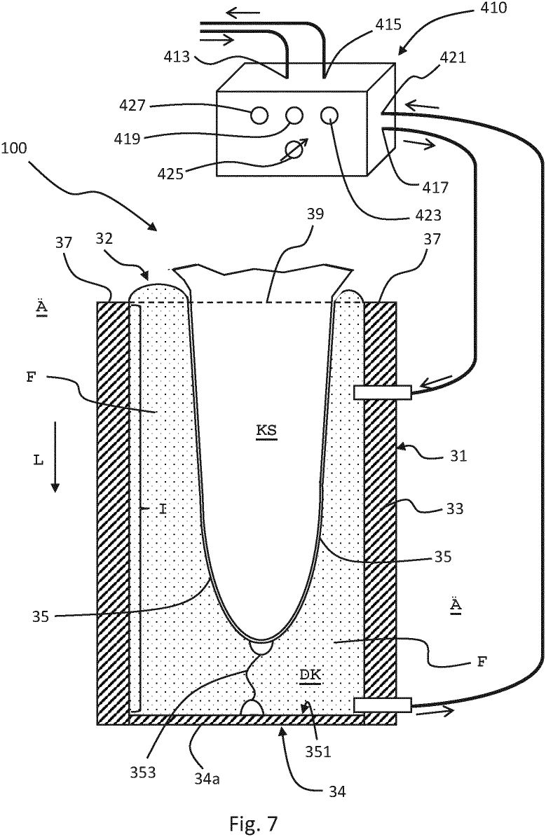

Shown on the left in FIG. 6, a limb stump KS, of the patient P, only partially represented, may, when measured, have the measurement results shown in the table on the right in FIG. 6. Column B shows the measurement results determined on the limb stump KS of patient P and indicates the respective measured circumference (in cm) at a distance of e.g. 6 cm, 9 cm and 12 cm from a predetermined reference point or reference cross-section (column A). The values of column B are also considered herein to be actual data of patient P. They were measured at the positions of the limb stump KS indicated in column A prior to manufacturing the prosthetic shaft 2, preferably with the herein disclosed impression device or device 100, as shown for example in FIG. 7 or disclosed herein.

When examining a plurality of patients of a collective with comparable limb stumps and/or comparable patient data, e.g. before the start of the method according to the present invention for creating and/or in the course of the method for creating a data collection, there were values measured, i.e. actual data was determined on the collective, which - here exemplarily - indicates the cross-sections of the limb stump, e.g. 6 cm, 9 cm and 12 cm, at later, defined wearing timepoints, at which preferably measurements were taken again by the impression device - or by an identically constructed type of device. They are indications or clues of what the limb stump KS of patient P will probably look like at the reference points or reference cross sections mentioned in column A at later wearing timepoints. Thus, the numerical values in column C, for example, indicate which circumference values will be present at the positions named in column A at a first wearing timepoint, for example 3 months after the timepoint of creation of the prosthesis, since there were similar values present in the collective; if necessary, they are actual data determined on the collective, alternatively related to the limb stump KS of the patient P. Thus, said numerical values in column C preferably indicate which measured values the limb stump KS of patient P would presumably assume, since these measured values have already applied to a sufficiently large collective, for instance by reflecting the changes of the measured values over three months - preferably each determined by or using the impression device - being observed in the collective and, if necessary, have already been determined after having been mathematically adjusted to the measured actual data of patient P.

Column D gives circumferential values, for which one may assume, due to the previous measurements on the above-mentioned patient collective, that the actual limb stump shown on the left in FIG. 6 will also assume these (or very similar) values/dimensions at the positions specified in column A at the second wearing timepoint, for example after about 6 months.

FIG. 6 shows, in other words, a reference source on the right. The columns C and D thereof show which geometric data a limb stump, which at the time of initial fitting with a prosthesis (e.g. at the timepoint of determination) has the values of column B at the positions specified in column A, will according to assumption or experience have at the, herein exemplarily considered, first and second wearing timepoints, namely the values of columns C and D. Reading them out may represent a determination in the sense of the present invention.

If patient P is a diabetic (as an example for the patient data), the values in columns C and D could already be determined from a collective that also consisted of diabetics. According to the inventor’s experience, their limb stumps regularly change more slowly between different measurement timepoints than it is the case in patients who, have lost e.g. their lower leg in an accident. If patient P were an athlete, columns C and D might show different values.

Furthermore, the values in columns C and D can already take into account how large the values in column B are. If the specific patient P had shown greater actual values than those noted in column B, the values C and D could also have been different, in particular greater.

FIG. 6 serves as an example. The present invention is not limited to considering the circumference as a measured or determined geometric datum. The use of other actual data and patient data is, additionally or alternatively, in combination with each other or alone, likewise encompassed by the present invention.

Instead of taking them from a reference source, geometric data may, according to the present invention, be determined based on a provided set of data at the moment of determination.

Although the present invention is described or discussed herein in a number of passages and in particular on the basis of the exemplary figures using the example of the limb stump of a lower extremity (thigh, lower leg, foot), the present invention is by no means limited to the fitting of a limb stump of the lower extremity. According to the present invention, what is described herein also applies without restriction to the fitting of the upper extremity (upper arm, lower arm, hand) as well as to the products proposed for fitting, such as prosthetic shaft, inner shaft, outer shaft and extensions.

FIG. 7 shows an impression device or device 100 (both terms may designate the same device, i.e. they are interchangeable herein) cut longitudinally or in the longitudinal direction (i.e. with reference to FIG. 7 from top to bottom), which device 100 may be used according to the present invention, wherein said device 100 is shown in an exemplary embodiment e.g. of the method for manufacturing or for the planning of manufacturing and/or in an exemplary embodiment of the method for creating a data collection.

The device 100 comprises at least one pressure container 31, which in turn comprises or, consists of, a wall 33 and a membrane 35.

The pressure container 31, which may be purely optionally cylindrical as shown in FIG. 7, optionally comprises a first end face 32 (top in FIG. 7) and a second end face 34 (bottom in FIG. 7). In the exemplary embodiment of FIG. 7, the second end face 34 is fluid-tight sealed from an exterior Ä by a bottom plate or bottom surface 34a. The bottom surface 34a may be made of the same material as the wall 33.

The membrane 35 separates a fluid chamber or pressure chamber DK of the pressure container 31 fluid-tight from an exterior of the fluid chamber or pressure chamber DK, or for example from the exterior Ä, i.e. an environment of the pressure container 31, or, as shown in FIG. 7, from a limb stump KS introduced in or surrounded by the membrane 35.

The membrane 35 may be fluid-tight connected to the pressure container 31 at an upper edge 37 of the wall 33 which is often ring-shaped, rectangular, square or differently circumferential, or at another site.

The upper edge 37 lies here exemplarily in a plane in which an insertion opening 39 of the pressure container 31 lies, or delimits this on its circumference. The insertion opening 39 lies in the plane indicated by the dashed line.

The insertion opening 39 serves for inserting the limb stump KS, which is wrapped in a moist plaster bandage, into an interior I of the pressure container 31.

The interior I is the volume of the pressure container 31 delimited by the wall 33. It extends from the second end face 34, which is sealed fluid-tight with the bottom surface 34a, to the insertion opening, designated with reference numeral 39 and indicated by a dashed line.

The pressure chamber DK is filled with a fluid, here exemplarily with a liquid F indicated by dots. A filling with gas is also contemplated by the present invention.

In FIG. 7, the device 100 is shown in a state in which the extremely schematically indicated limb stump KS of the standing patient is inserted into the interior I such that it is, at least in its distal section, surrounded by the membrane 35. The membrane 35 rests against the plaster bandage on the limb stump KS like a second skin, wherein further layers such as liners or similar may be provided between the plaster bandage and the membrane 35.

The limb stump KS is preferably loaded with the full body weight of the standing patient. Knowing the volume of the interior I or of the pressure container 31, the amount of liquid F is preferably dimensioned such that the limb stump KS may enter the pressure container 31 through the insertion opening 39 at least to such a depth that the entire surface of the plaster bandage, however though as far as it is relevant for the impression, is in contact with the membrane 35. At the same time, the amount of liquid F is dimensioned such that the distal end of the limb stump KS (bottom of FIG. 7) preferably does not touch or rest on the bottom of the pressure container 31. This ensures that it is the pressure of the fluid on which the patient rests with the inserted limb and that the plaster bandage experiences the same pressure at every point by the membrane 35.

It can be seen from FIG. 7 that when no limb stump KS is inserted into the pressure container 31, the membrane 35 floats or raises due to the pressure of the fluid, here the liquid, and a liquid level, not shown in FIG. 7, is reached. The shape of the membrane 35 shown in FIG. 7 thus represents that shape which the membrane 35 assumes under load when it rests against the inserted limb stump KS and is “carried along” by the latter - in the example of FIG. 7 - into the depth of the interior I in the direction of the bottom surface 4a.

It can also be seen from the figure that by preventing fluid exchange between the pressure chamber DK and the exterior Ä or by preventing fluid from escaping from the pressure chamber DK, the wall 33 and the membrane 35 enable the desired pressure builds up within the pressure chamber DK of the pressure container 31, but cannot escape from it or degrade or decrease.

As can be seen in FIG. 7, the pressure chamber DK is thus formed by the membrane 35 and by at least parts of the wall 33, to which here the bottom surface 34a of the end face 34 is also included.

In embodiments of the present invention other than the exemplary embodiment shown in FIG. 7, the pressure chamber DK may consist of or comprise a membrane closed all around, which may similar to a balloon or bladder lie in the interior I of the pressure container 31.

The same applies for the pressure container 31 shown in FIG. 7 or in one of the following figures as well as for the pressure container of any other embodiment according to the present invention, that it may comprise a non-circular, preferably an angular, cross-section instead of a circular cross-section. The cross-section may be e.g. rectangular, polygonal or square. The latter cross-sections may advantageously avoid or reduce artifacts that may be caused by the curved circumferential surface, in particular when measuring the limb stump KS inserted into the device 100 automatically, using a camera or similar as described herein.

The membrane 35 is optionally force-fit connected to a section 351 of the wall 33 or the end face 34, preferably in its distal middle or central section or area. In the embodiment shown in FIG. 7, the force-fit connection is effected by an optional connector 353, which extends from a distal end of the membrane 35 to the section 351, here purely optionally the bottom surface 34a of the pressure chamber DK.

The connector 353 may be a thread, as exemplarily shown in FIG. 7. Any other suitable connector, such as a tape, Velcro or similar, is also encompassed by the present invention.

The connector 353 holds the membrane 35 in a force-fit and/or form-fit and/or material-bond connection to the pressure container 31 or to the bottom surface 34a, preferably in the region of the second end face 34 of the pressure container 31, in particular in the region of the bottom surface 34a and preferably in the middle or center thereof.

It is preferred that the connector 353, at least when the device 100 is in use, i.e. with the limb stump KS inserted into the pressure chamber DK, allows flow around the distal end of the limb stump KS completely or substantially completely. A flow around the limb stump KS is thus advantageously still possible in FIG. 7 except for the area corresponding to the cross-section of the connector 353. In this way, buoyancy or the pressurization of the distal section of the limb stump KS, which is very important for readjusting or recreating the subsequent load situation in the finished shaft, may also take place by the fluid in an unchanged manner.

In addition to the optional connector 353 shown in FIG. 7, several or additional connectors may be provided. These may be connected to the bottom surface 34a like the connector 353. They may alternatively or additionally be connected to the pressure chamber DK or the pressure container 31 at a section of the wall 33 being different than the end face or bottom surface 34a. This also applies to the connector 353 shown in FIG. 7.

The connection between connector 353 on the one hand and wall 33 or bottom surface 34a on the other hand may - such as, independently therefrom, the connection between connector 353 on the one hand and membrane 35 on the other hand - be an adhesive connection, a screw connection, a plug-in connection, a snap-in connection, a latching connection or similar. It may be releasable or non-releasable.

The connector shown in FIG. 7 is preferably not elastically stretchable. It is preferably not stretchable.

The membrane 35 shown in FIG. 7 is also preferably non-stretchable and/or non-elastic in the longitudinal direction L.

In certain embodiments, “non-stretchable” or “non-elastic” means that the Young’s modulus of the relevant component (connector, membrane, fibers, etc.) is at least above 700 N/mm², preferably above 1000 N/mm², more preferably above 2000 N/mm².

In some embodiments, “non-stretchable” or “non-elastic” means that an elongation of the relevant component (connector, membrane, fibers, etc.) may not exceed 20%, preferably not more than 10%, preferably not more than 5%, more preferably not more than 2% of its length before the component tears or breaks.

The medical device 100 is preferably connected to a pressurization control device 410. The pressurization control device 410 is hereinafter abbreviated as the control device 410. It may be designed to regulate the pressure.

The control device 410 comprises a pressure reservoir connector 413, which may be designed as a connection to a pressure source, e.g. to a water line. Furthermore, in the embodiment shown here, the control device 410 comprises a separate discharge connector 415 for emptying the fluid from the pressure chamber DK. The discharge could alternatively take place e.g. via a multi-way valve which may be connected to the pressure reservoir connector 413.

The control device 410 of FIG. 37 further comprises an optional pressure-control valve arranged within the control device 401 (not shown in FIG. 7). The pressure-control valve may limit the pressure prevailing in the pressure reservoir connector 413, so that the pressure applied downstream of the pressure-control valve does not exceed a predeterminable value or a value optionally adjustable by the user. As a result, an excessively high pressure in the pressure chamber DK may advantageously be avoided or prevented, which, for example, might cause an excessive pressure load on the limb stump KS, and thus could cause damage and/or pain, as well as a distortion of the plaster impression. The pressure-control valve may purely exemplarily limit the pressure to max. 0.8 bar (800 hPa).

The control device 410 optionally further comprises a pressure chamber connection 417, which is connected to, or comprises, e.g. a regulating valve 419 for increasing the pressure in the pressure chamber DK. When the regulating valve 419 is actuated, e.g. manually, it can increase the pressure in the pressure chamber DK, e.g. gradually, up to a desired pressure or to the maximum pressure which is limited by the pressure-control valve.

Furthermore, the control device 410 optionally comprises a pressure chamber backflow connector 421, which is connected to, or comprises an additional regulating valve 423 for reducing pressure in the pressure chamber DK. When the additional regulating valve 423 is actuated, e.g. manually, it can reduce the pressure in the pressure chamber DK, e.g. gradually, as required. Once the manufacturing of the plaster impression has been completed, i.e. after a predetermined drying or curing time, the pressure in the pressure chamber DK can be reduced to the point where the patient may pull the limb stump KS out of the device 100.

In the illustrated embodiment, the control device 410 further comprises an optional inlet pressure display 425 and a likewise optional shutdown device 427. The inlet pressure display 425 may be referred to as a manometer and may be provided as an additional display, for example as a safety display or redundancy arrangement for monitoring the pressure in the pressure chamber DK. The inlet pressure display 425 is arranged, e.g., with, or in, a connection line in the control device 410 between the regulating valve 419 (for the pressure increase in the pressure chamber DK) and the pressure chamber connection 417.

Emptying the pressure chamber DK using the pressure chamber backflow connector 421 and the outlet connection 415, controlled by the additional regulating valve 423, may be effected in various ways. For example, a suction pump may be connected - for example downstream of the discharge connection 415 - which, when the additional regulating valve 423 is opened, sucks fluid F from the pressure chamber DK and, if necessary, until empty. Likewise, an optional venturi nozzle or a Venturi tube for sucking or draining the fluid F from the pressure chamber DK may be connected or is connected, for example, downstream of the discharge connector 415. A Venturi nozzle may, for example, be connected to an external water connection, for example to a water tap on a washbasin. If the water tap is opened and water flows through the optional Venturi nozzle, a vacuum is generated in a line connected to the outlet connection 415, and fluid F, with the additional regulating valve 423 being open, is sucked. A Venturi nozzle advantageously does not require any electrical connection, as would for example be necessary for a suction pump.

The exemplary embodiment of the control device 410 described here, advantageously does not require any electrical components and thus no power supply and no power connection. Embodiments which differ to this are likewise encompassed by the present invention.

In embodiments of the present invention other than those described herein, the pressurization control device 410 is a device for applying pressure to a fluid reservoir, for instance a supply reservoir.

For this purpose, it is contemplated to provide, e.g., a mechanical or hydraulic pressing device by which the fluid may be discharged from a supply reservoir or from the pressurization control device 410 under a desired pressure or in any case a sufficient pressure.

Such a mechanism may include a crank mechanism, a pressing or clamping mechanism having clamping surfaces, a foot actuating device the construction of which corresponds or is similar to a bellows or to a bicycle pump. Using such a pressurization control device 410, the required pressure may be achieved on the one hand, the user of the device 100 according to the present invention is on the other hand not dependent on external pressure sources, such as water connections or similar.

The pressurization control device 410 may be operated electrically. However, this is not required. It can be provided to be able to operate without power supply.

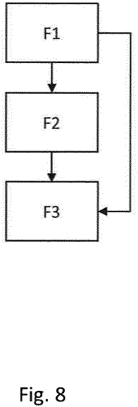

FIG. 8 shows the method according to the present invention for manufacturing or planning the manufacturing of a prosthetic shaft 2, an inner shaft 6 and/or an outer shaft 4 and/or an extension 12, 14, 20 of the prosthetic shaft 2, schematically simplified in an exemplary embodiment. The prosthetic shaft is provided in order to receive a limb stump KS (see FIG. 7).

The method according to the present invention encompasses determining data, in particular geometric data, or providing data, in particular geometric data. In this, preferably such data is determined, in particular geometric data, which will describe, determine or co-determine the shape of the limb stump KS at least at one timepoint in the future. Preferably, data is hereby determined which, at least at the first wearing timepoint, should co-determine or define the shape of the prosthetic shaft 2, the inner shaft 6 or the outer shaft 4 or of the extension(s) 12, 14, 20 (or the shape which these should then have). The data or geometric data may be predicted values or measurements, estimated values or expected values, in particular about future shapes. The data or geometric data may have been created based on measurements done on the collective or may be derived based thereon as disclosed herein. The measurements on the collective may have been collected using the device or impression device disclosed herein, see e.g. FIG. 7.

The method optionally further encompasses manufacturing the prosthetic shaft 2 or sections thereof as method step F2. This is done based on the determined data and/or geometric data.

Alternatively or in addition to the aforementioned manufacturing of the prosthetic shaft 2 or sections thereof, the method encompasses as step F3 creating at least one control signal, in particular creating a control file with control signals, based on which a manufacturing machine may (e.g. directly or indirectly) perform steps for manufacturing the prosthetic shaft 2, the inner shaft or outer shaft or the extension 12, 14, 20 and/or create at least one of the aforementioned devices or parts thereof.

Thereby, the timepoint of creation (also: creation timepoint) is preferably earlier than the first wearing timepoint.

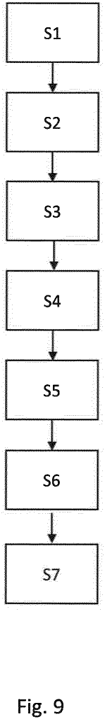

FIG. 9 shows the method according to the present invention for creating a data collection or for collecting measurements of the limp stump schematically simplified in an exemplary embodiment.

The method for creating a data collection according to the present invention encompasses as step S1 providing a device 100 for example the impression device disclosed herein for use in manufacturing a plaster impression or creating a data model of a limb stump KS. The device 100 comprises a container or pressure container 31 having a pressure chamber DK for receiving pressurized fluid F, preferably a liquid (see FIG. 7). The pressure container 31 comprises a wall 33 made of a first material and a fluid-tight membrane 35 made of a second material.

As an optional step S2, the method according to the present invention encompasses providing a pressurization device and/or a pressurization control device 410 for setting, effecting and/or maintaining a pressure prevailing within the pressure chamber DK.

Step S3 of the method according to the present invention is a measuring of actual data of the limb stumps KS, preferably at always the same, predetermined positions, circumferences or sites of or on the limb stumps, of a plurality of patients or a collective of patients. The measuring is carried out, respectively, at least at one first measurement timepoint by the device 100. Measuring on the collective of patients may hereby be done using the device 100, by a measuring device contained in or connected to said device 100 or by hand. The measurement may be done directly, e.g., on the limb stump of the patients of the collective, and/or indirectly, e.g., on positive or negative models, impressions, plaster impressions, or prosthetic shafts, etc., being respectively created by the device 100 or geometric data obtained therewith for the patients of the collective.

The measuring of actual data of the limb stumps, respectively, at least at one second measurement timepoint, which lies after the first measurement timepoint, is encompassed as a further step S4 by the method according to the present invention. The measuring is carried out here preferably also by the device 100, or as indicated above, and preferably at the same positions or sites of the limb stump just like the measuring carried out at the first measurement timepoint.

As step S5, the method optionally encompasses a determination or an association of patient data to the measured actual data of the patient collective which was measured at the first measurement timepoint or at the second measurement timepoint. The measured actual data, which was measured at the at least two measurement timepoints, is thus associated to the same patient data, respectively.

A subsequent classification of the actual data measured at least at the first measurement timepoint and the actual data measured at least at the second measurement timepoint based on the patient data associated to them is also encompassed as a further optional step S6 by the method according to the present invention.

In a step S7 of the method according to the present invention for creating a data collection, the classified actual data is stored, based on its classification, in a suitable storage medium, such as a database, e.g. in order to be able to (re)access it within the scope of the method according to the present invention for manufacturing or planning the manufacturing of a prosthetic shaft 2, inner shaft 6, outer shaft 4 and/or of an extension 12, 14, 20 of the prosthetic shaft (see also FIG. 8). Alternatively or additionally, the data can be evaluated and/or further processed by suitable devices.

List of reference numerals

| | |
|---|---|
| 2 | prosthetic shaft |
| 4 | outer shaft |
| 5 | inlet opening |
| 6 | inner shaft |
| 7 | slot |
| 8 | distal end of the outer shaft |
| 10 | column-like or columnar component |
| 12 | extension |
| 14 | extension |
| 18 | wall |
| 20 | extension |
| 22 | edge |
| 24 | femur |
| 26 | empty space |
| 28 | empty space |
| 31 | pressure container |
| 32 | first end face |
| 33 | wall |
| 34 | second end face, may optionally be closed or |
| | sealed with the base surface |
| 34 a | base surface |
| 35 | membrane |
| 37 | upper edge |
| 39 | insertion opening |
| 100 | device |
| 200 | computing system |
| 210 | calculation device |
| 220 | reference source |
| 230 | input device |
| 240 | output device |
| 250 | manufacturing machine |
| 260 | pedometer or mobility sensor |
| 351 | lower section of the wall |
| 353 | connector |
| 410 | pressurization control device pressurization |
| | device |
| 413 | pressure reservoir connector |
| 415 | discharge connector |
| 417 | pressure chamber connector |
| 419 | regulating valve |
| 421 | pressure chamber backflow connector |
| 423 | additional regulating valve |
| 425 | inlet pressure display |
| 427 | emergency stop device |
| Ä | exterior of the pressure container |
| DK | pressure chamber of the pressure container |
| F | fluid or liquid |
| I | interior of the pressure container |
| KS | limb stump |
| L | longitudinal direction |

1. A method for manufacturing or for planning the manufacturing of a prosthetic shaft, an inner shaft, an outer shaft and/or an extension of the prosthetic shaft, wherein the prosthetic shaft is provided for receiving a limb stump of a patient,
encompassing the steps:
determining geometric data or providing geometric data, wherein the geometric data will co-determine the shape of the limb stump at least at a first wearing timepoint of the prosthetic shaft, the inner shaft, the outer shaft, or of the extension; and either
creating, based on the determined geometric data, the prosthetic shaft, the inner shaft or outer shaft, or the extension, or creating sections thereof, based on the geometric data, or
creating a control file having control signals upon which a manufacturing machine may execute steps for creating the prosthetic shaft, the inner shaft, the outer shaft or the extension, wherein said creating takes place at a creation timepoint prior to the first wearing timepoint, and wherein the geometric data is not data measured on the patient.

2. The method according to claim 1, further encompassing the step of
reading a pedometer or mobility sensor which records the steps or mobility of the patient.

3. The method according to claim 1, wherein the geometric data is predicted data, data of variable dimensions of the limb stump-fKSi, data which is subject to, or caused by, post-operative changes over time and changes caused by a previous surgical operation, and/or data which does not represent or reflect the shape of the limb stump at the timepoint of determining data or at the creation timepoint, and/or wherein the geometric data is not actual data and/or measured data of the patient.

4. The method according to claim 1, wherein the determining of the geometric data takes into account patient data which reflects the, or at least one, in particular momentary, health condition or finding of the patient at the timepoint of the determining and/or at a past timepoint and/or wherein the geometric data was collected from a collective of patients having this health condition or finding.

5. The method according to claim 1, wherein the geometric data is or was collected by or using a device on a collective of patients, wherein the device is suitable and/or provided for use in manufacturing a plaster impression or creating a data model of the limb stump and comprises a pressure container with a pressure chamber for receiving pressurized fluid, preferably a liquid, wherein the pressure container comprises a wall made of a first material and a fluid-tight membrane made of a second material.

6. The method according to claim 1, wherein the geometric data is or encompasses the result of an estimation, a readout from a reference source and/or a calculation based on an algorithm.

7. The method according to claim 1, wherein at least 3 days, preferably at least 10, 20, 30, 60, 90 days, in particular at least 180 days, 12 months or 24 months lie between the creation timepoint and the first wearing timepoint.

8. The method according to claim 1, wherein the geometric data additionally also encompasses data which will co-determine the shape of the limb stump at least at a second wearing timepoint which is after the first wearing timepoint, wherein at least 3 days, preferably at least 10, 20, 30, 60, 90 days, in particular at least 180 days, 12 months or 24 months lie between the first wearing timepoint and the second wearing timepoint.

9. The method according to claim 1, wherein in the step of creating, a prosthetic shaft, an inner shaft, an outer shaft and/or at least one extension or a corresponding control file for the manufacturing machine is also created based on the geometric data which will co-determine the shape of the limb stump at least at the second wearing timepoint.

10. The method according to claim 1, wherein the inner shaft serves to receive at least portions of the limb stump and in turn is provided to be at least partially received in an interior of the outer shaft.

11. The method according to claim 1, wherein the extension is an inlay, a pad, a pressure insert, a compression insert, a stocking with different wall thicknesses or a double-walled stocking with at least one insert inserted between its layers.

12. The method according to claim 1, wherein the manufacturing machine is a printer, a 3D printer, a casting device, a milling machine, a rapid prototyping device, a CNC milling machine, a CAD milling machine, a thermoforming device, or an injection device, configured to create the prosthetic shaft, the inner shaft, the outer shaft or the extension or sections thereof based on the control signals.

13. (canceled)

14. A kit with one element from a group consisting of prosthetic shaft, inner shaft, outer shaft or at least of one extension, each manufactured according to the method according to claim 1.

15. The kit according to claim 14, wherein at least a first element of the group was created based on the geometric data which co-determines the shape of the limb stump at the first wearing time, and wherein at least a second element of the group was created based on the geometric data which co-determines the shape of the limb stump at the second time.

16. The kit according to claim 14, further comprising at least one further element of the group which consists of prosthetic shaft, inner shaft, outer shaft or at least one extension, wherein said further element is generated based on actual data and/or measured data which co-determines the shape of the limb stump at the timepoint of creation.

17. (canceled)

18. (canceled)

19. (canceled)

20. (canceled)

21. A method for creating a data collection, encompassing the following steps:
providing a device for use in manufacturing a plaster impression or creating a data model of a limb stump-fKSi, comprising a container or pressure container having a pressure chamber for receiving pressurized fluid, preferably a liquid, wherein the pressure container comprises a wall made of a first material and a fluid-tight membrane made of a second material;
providing a pressurization device and/or a pressurization control device for setting a pressure to prevail within the pressure chamber;
measuring actual data of the limb stumps of a plurality of patients respectively at least at one measurement timepoint by or using the device;
measuring actual data of the limb stumps respectively at least at one second measurement timepoint, which lies after the first measurement timepoint, by or using the device;
associating patient data to both the actual data which was measured at the first measurement timepoint as well as to the actual data which was measured at least at the second measurement timepoint;
classifying the actual data measured at the first measurement timepoint and the actual data measured at the second measurement timepoint based on the patient data associated therewith;
saving and/or evaluating and/or further processing of the classified actual data, based on the classification thereof, in a suitable storage medium, for instance a database.

22. (canceled)

23. (canceled)

24. A set comprising
a computing system programmed to carry out the method according to claim 1 ; and
a device for use in manufacturing a plaster impression, or creating a data model, of the limb stump-fKSi, which comprises a container or a pressure container having a pressure chamber for receiving pressurized fluid, preferably a liquid, wherein the pressure container comprises a wall made of a first material and a fluid-tight membrane made of a second material.

25. The set according to claim 24, further comprising
a pedometer or mobility sensor.

26. The set according to claim 24, wherein the computing system and the device are in signal communication with each other in order to transmit the actual data measured or collected by the device to the computing system, or are prepared, configured and/or programmed for this.

27. The set according to claim 24, wherein the computing system and the pedometer or mobility sensor are in signal communication with each other in order to transmit the data measured or collected by the pedometer or mobility sensor to the computing system, or are prepared, configured and/or programmed to be for this.
